(19) United States
(12) Patent Application Publication
(10) Pub. No.: US 2008/0154308 A1
(43) Pub. Date: Jun. 26, 2008

(54) Spinal fixation system

(75) Inventors: Michael C. Sherman, Memphis, TN (US); Roy K. Lim, Memphis, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: Warsaw Orthopedic, Inc.

(21) Appl. No.: 11/643,283

(22) Filed: Dec. 21, 2006

(51) Int. Cl. A61B 17/58; A61B 17/56

(52) U.S. Cl. 606/265; 606/246; 606/305

(57) ABSTRACT

A spinal implant system comprises at least two fixation elements, each including a threaded bone-engaging portion, and at least two corresponding receiver members, each including two branches defining a U-shaped channel configured to receive an elongated member. The receiver members are positionable in both open and closed positions. The system further comprises at least one elongated member configured to be positioned in the channels. Additionally, the system comprises at least two snap-fit caps configured to snap on to the receiver members near the top surfaces of the corresponding branches to substantially lock the receiver members in closed positions and capture the elongated member in the channels.

The present disclosure broadly concerns spinal fixation systems and relates to a system involving an orthopedic implant with a snap-fit cap to secure an elongated member, such as a spinal rod, relative to the implant. The system can be useful for correction of spinal injuries or deformities.

The present disclosure generally relates to a radiolucent, smoothly-contoured, motion-preserving rod and screw system, and more specifically, but not exclusively, concerns a radiolucent system involving a cap configured to snap-fit to a receiver member, such as a bone screw head, to secure a spinal rod therein. Additionally, the present disclosure concerns a spinal fixation system involving a resilient bumper engaging at least one elongated member and positioned between two fixation elements to maintain a separation distance between the fixation elements, prevent translation of the elongated member, and/or provide resiliency to the system.

In the realm of orthopedic surgery, it is well known to use implants to fix the position of bones. In this way, the healing of a broken bone can be promoted, and malformations or other injuries can be corrected. For example, in the field of spinal surgery, it is well known to place such implants into vertebrae for a number of reasons, including (a) correcting an abnormal curvature of the spine, including a scoliotic curvature, (b) to maintain appropriate spacing and provide support to broken or otherwise injured vertebrae, and (c) perform other therapies on the spinal column.

Typical implant and connection systems include several pieces, which commonly are useful and may be associated with only specific other pieces. Bone screws, hooks, and clamps are well known as fixation devices, which are connected or adjoined to a particular bone as a connection between the bone and the connection system which can include a support and/or stabilizing member such as a spinal rod. In such a system, a series of two or more bone screws may be inserted into two or more vertebrae to be instrumented. A spinal rod is then placed within or coupled to the screws, or is placed within a connecting device that links the rod and a screw, and the connections are tightened. In this way, a rigid supporting structure is fixed to the vertebrae, with the rod providing the support that promotes correction or healing of the vertebral malformation or injury by keeping the vertebrae in a particular position.

A multitude of spinal fixation systems exist; however, the systems can be difficult to assemble and secure, and can cause tissue irritation and/or damage to the surrounding area. Therefore, a need exists for improved spinal fixation systems.

### BRIEF DESCRIPTION OF THE DRAWINGS

FIG. 1 is a perspective view of a spinal fixation system according to an embodiment of the present disclosure.

FIG. 2 is another perspective view of a spinal fixation system according to an embodiment of the present disclosure.

FIG. 3 is a perspective view of a component of the spinal fixation system according to the embodiment of FIG. 1.

FIG. 4 is a perspective view of a component of the spinal fixation system according to the embodiment of FIG. 1.

FIG. 5 is a perspective view of a component of the spinal fixation system according to the embodiment of FIG. 1.

FIG. 6 is a perspective view of a component of the spinal fixation system according to the embodiment of FIG. 1.

FIG. 7 is a perspective view of a component of the spinal fixation system according to the embodiment of FIG. 1.

FIG. 8 is a cross-sectional view of components of the spinal fixation system according to the embodiment of FIG. 1.

FIG. 9 is a perspective view of the spinal fixation system according to the embodiment of FIG. 1.

FIG. 10 is a perspective view of the spinal fixation system according to the embodiment of FIG. 1.

FIG. 11 is a cross-sectional view of components of the spinal fixation system according to the embodiment of FIG. 1.

FIG. 12 is a perspective view of the spinal fixation system according to the embodiment of FIG. 1.

FIG. 13 is a perspective view of a spinal fixation system according to another embodiment of the present disclosure.

FIG. 14 is a perspective view of components of the spinal fixation system according to the embodiment of FIG. 13.

FIG. 15 is a perspective view of a component of the spinal fixation system according to the embodiment of FIG. 13.

FIG. 16 is a perspective view of a component of the spinal fixation system according to the embodiment of FIG. 13.

FIG. 17 is an exploded, perspective view of components of the spinal fixation system according to the embodiment of FIG. 13.

FIG. 18 is a perspective view of a component of the spinal fixation system according to the embodiment of FIG. 13.

FIG. 19 is a perspective view of components of the spinal fixation system according to the embodiment of FIG. 13.

FIG. 20 is a perspective view of components of the spinal fixation system according to the embodiment of FIG. 13.

FIG. 21 is a perspective view of components of the spinal fixation system according to the embodiment of FIG. 13.

### DESCRIPTION OF THE ILLUSTRATED EMBODIMENTS

For the purpose of promoting an understanding of the principles of the disclosure, reference will now be made to the embodiments illustrated in the drawings and specific language will be used to describe the same. It will nevertheless be understood that no limitation of the scope of the disclosure is thereby intended, such alterations and further modifications in the illustrated devices, and such further applications of the principles of the disclosure as illustrated therein, being contemplated as would normally occur to one skilled in the art to which the disclosure relates.

The present disclosure provides spinal fixation systems useful for correction of spinal injuries or deformities. The systems can be radiolucent, smoothly-contoured, motion-preserving rod and screw systems. In certain embodiments, the systems include a fixation element, such as a bone screw, with a receiver member having sides that can be opened or expanded and closed or contracted. A snap-fit cap can be used to secure an elongated member, such as a spinal rod, relative to the fixation element. In addition, a resilient bumper can engage at least one spinal rod and be positioned between two bone screws to maintain a separation distance between the bone screws, prevent translation of the spinal rod, and/or provide resiliency to the system. Methods of assembling and implanting the systems described herein are also provided.

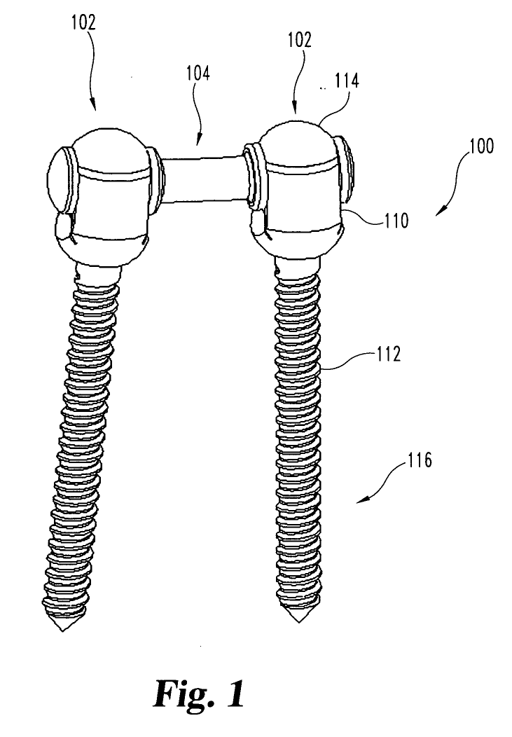

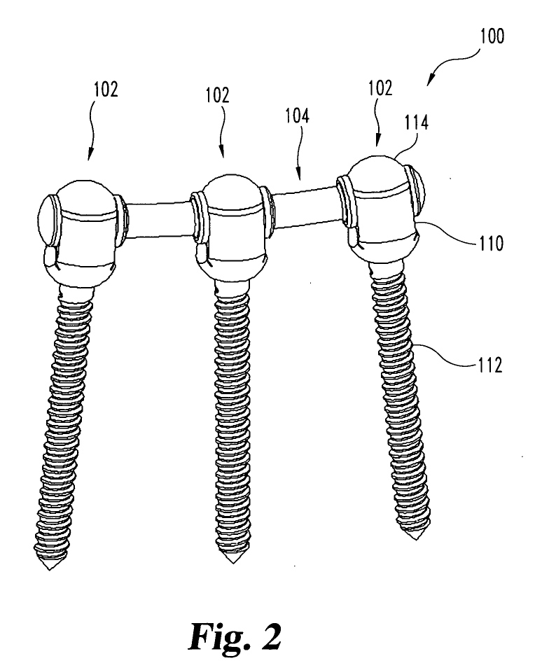

Referring generally to FIGS. 1-12, there is shown an embodiment of a spinal fixation system 100. In this embodiment, system 100 includes one or more implants 102, such as a multi-axial bone screw, and an elongated member 104, such as a spinal rod. Implant 102 can be inserted into a vertebra of a spinal segment of a patient and member 104 can be utilized to maintain one or more vertebrae or spinal segment(s) at a desired position. It should be appreciated that the implant utilized in conjunction with system 100 can be any appropriate bone anchor or bone-engaging mechanism, such as a hook, clamp or monoaxial screw. Additionally, it should be appreciated that the elongated member utilized in conjunction with system 100 can be any appropriate elongated member such as a bar, connector, or other orthopedic construct. Further, the elongated member may have one of a number of desired lengths.

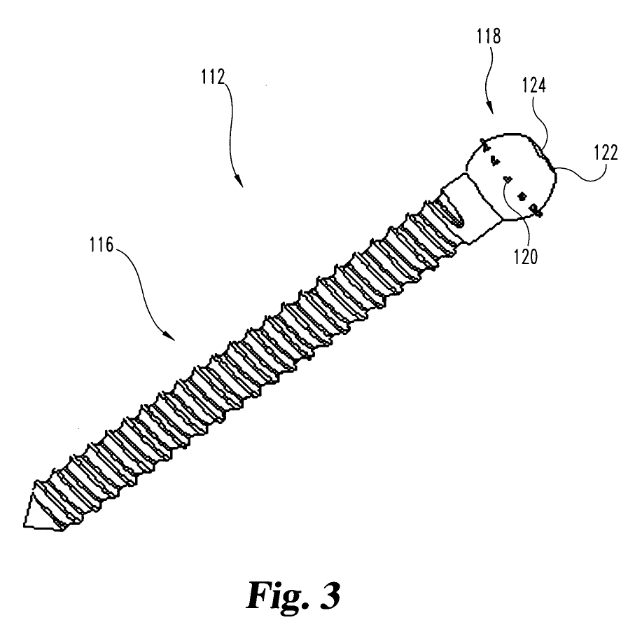

In the illustrated embodiments, implant 102 includes a connector or receiver member 110 in engagement with a fixation element 112 and a snap-fit cap 114. As illustrated in FIG. 3, fixation element 112 has a threaded bone-engaging portion 116 configured to engage bone and a head portion 118 received in receiver member 110. Bone engaging portion 116 can include coarse threads readily adapted for solid fixation within the cancellous bone of a vertebral body and can terminate in a tapered tip to assist in the gradual engagement and advancement of the threads into the vertebral body. Head portion 118 can include sharp projections or barbs 120 to assist in securing fixation element 112 to receiver member 110, as will be discussed in greater detail. In the illustrated embodiment, barbs 120 are substantially pyramidal with sharp points, and equally positioned about a circumference of head portion 118 and generally along radii of head 118 (and perhaps around a great circle of head 118). However, it should be appreciated that barbs 120 can be sized, shaped and positioned differently relative to head portion 118. In the illustrated embodiment, head portion 118 includes a generally spherical surface 122 and defines a tool-receiving bore 124. Tool-receiving bore 124 is configured to receive the head of a driving tool, such as a screwdriver, to engage fixation element 112 to bone. It should be appreciated that head portion 118 can be configured differently than as in the illustrated embodiment.

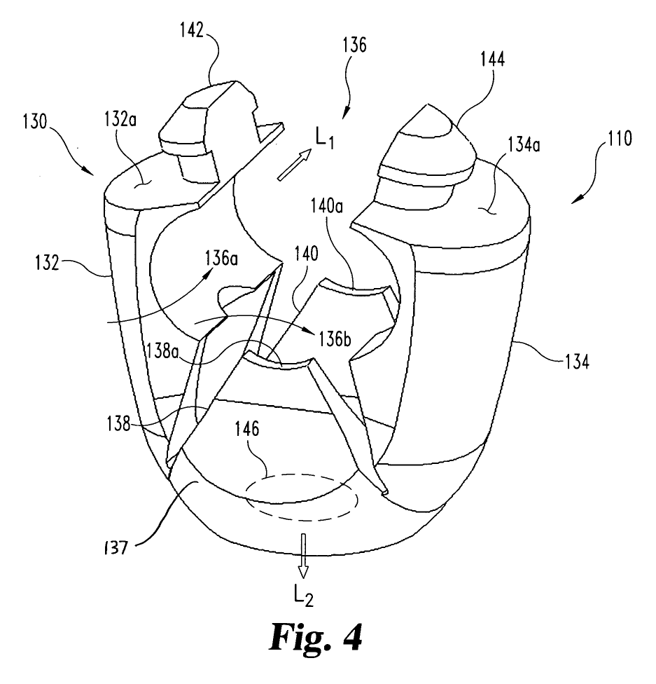

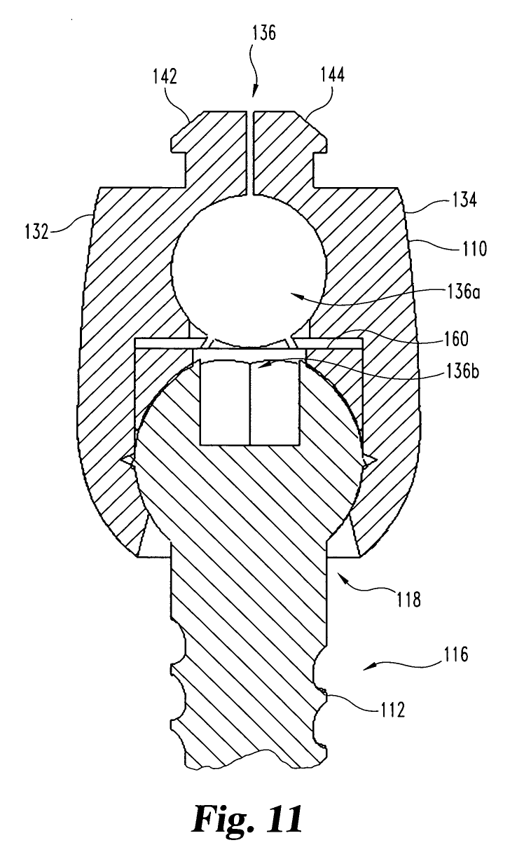

Referring to FIG. 4, there is shown an embodiment of receiver member 110 configurable in both an open position (see FIG. 8) and a closed position (see FIG. 11). The illustrated embodiment of receiver member 110 includes two opposing branches 132 and 134 defining a channel 136. In certain embodiments, channel 136 can include an upper portion 136a configured to accommodate elongated member 104 and a lower portion 136b configured to accommodate head portion 118 of fixation element 112. Channel 136 is substantially U-shaped and includes a longitudinal axis L1, with member 104 generally extending along longitudinal axis L1 when positioned in channel 136. In certain embodiments, receiver member 110 is smoothly-contoured on an outer surface thereof to reduce tissue irritation and/or other tissue damage upon implantation of receiver member 110 adjacent a spinal segment of a patient. In the illustrated embodiment, receiver member 110 is substantially egg-shaped; however, it should be appreciated that receiver member 110 can be sized and shaped differently than as illustrated.

Receiver member 110 includes a base portion 137 having two opposing branches 138 and 140 (which are shorter than branches 132 and 134 in this embodiment) further defining lower portion 136b of channel 136. Branches 132 and 134 are connected at a lower portion to base 137 so that branches 132 and 134 can pivot with respect to base 137 to open and close channel 136. In this embodiment, branches 132 and 134 and base 137 are a single unit, and the pivoting relationship arises from flexibility in receiver member 110 between branches 132 and 134 and base 137. In other embodiments, one or both of branches 132 and 134 may be separate from base 137 or other part of receiver member 110, and linked by tongue-and-groove, hinged, or other pivoting connection. A closed position of branches 132 and 134 is where branches 132 and 134 are substantially adjacent or abutting each other and base 137, and channel 136 is substantially closed. An open position of branches 132 and 134 is where they are positioned at least somewhat away from each other and base 137, and channel 136 is substantially open (e.g. to place elongated member 104 down into channel 136 as viewed in FIG. 4).

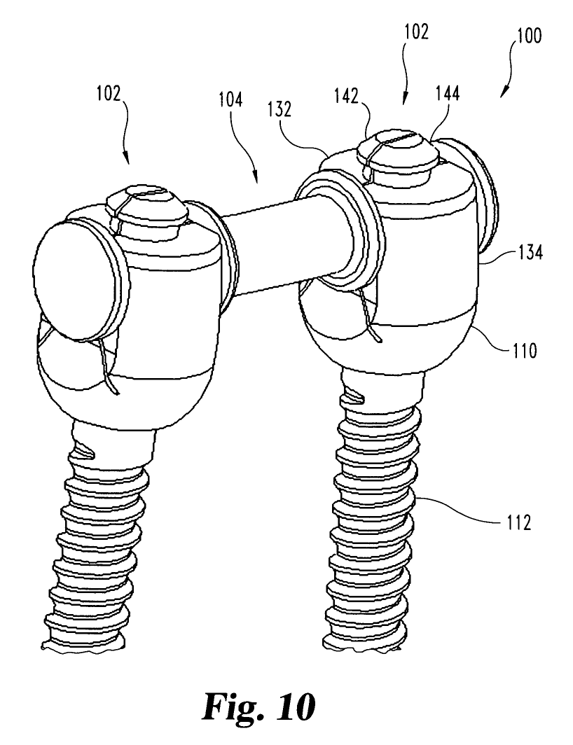

Branches 132, 134, 138 and 140 can include upper surfaces 132a, 134a, 138a and 140a, respectively. In certain embodiments, upper surfaces 132a and 134a can include snap projections 142 and 144, respectively, configured to engage with snap-fit cap 114 to secure elongated member 104 in upper portion 136a of channel 136. In the illustrated embodiment, snap projections 142 and 144 are mirror images of each other and include half-circular cross-sectional shapes in a direction generally parallel to rod axis L1, and substantially conical upper surfaces. Additionally, when receiver member 110 is in a closed position (see FIG. 10), snap projections 142 and 144 are positioned adjacent each other and are generally cylindrical in shape in combination, with sections of varying diameter. However, it should be appreciated that snap projections 142 and 144 can be configured differently. Moreover, it is contemplated that other types of mechanisms may be utilized such that cap 114 snaps to receiver member 110. Additionally, as illustrated, upper surfaces 138a and 140a or branches 138 and 140 can be generally concave to fittingly engage the convex outer surface of elongated member 104.

In certain embodiments, the width of upper portion 136a of channel 136 is approximately the same as or slightly larger than the diameter of member 104, which may allow for easier insertion of member 104 in upper portion 136a, may allow for compensation for contouring of member 104, and also may allow for use of a range of member diameters or widths with the same channel 136. In other embodiments, upper portion 136a of channel 136 is configured to allow a snap-fit of member 104 therein. As illustrated in FIG. 4, channel 136 is at least partially open along axis L1 to accommodate top loading of member 104. It should be appreciated that channel 136 can be configured and shaped differently than as illustrated in the accompanying figures.

Receiver member 110 may also define a hole 146 extending through base 137 and in communication with channel 136, with hole 146 being configured for at least partial passage of fixation element 112 during assembly of system 100. In the illustrated embodiment, hole 146 extends through receiver member 110 along longitudinal axis L2 and has a generally circular cross-sectional dimension. In certain embodiments, hole 146 is substantially perpendicular to channel 136 and substantially parallel to branches 132, 134, 138 and 140. However, it should be appreciated that hole 146 can include other cross-sectional shapes, orientations and dimensions. Channel 136 and hole 146 could be positioned differently relative to each other in a manner that would maintain the functions of system 100.

In the illustrated embodiment, receiver member 110 and fixation element 112 of implant 102 are two separate components. However, in other embodiments, the implant could include an integral bone screw device having a threaded post and a bone screw head to receive the elongated member. Additionally, it should be appreciated that the implant could include a multi-axial bone screw, with a bone screw head positionable at a plurality of angular positions relative to the bone screw threaded post.

In certain embodiments, system 100 can optionally include a crown member 160, as illustrated in FIG. 5. Crown member 160 may be positionable adjacent head portion 118 of fixation element 112 and configured to be received in lower portion 136b of channel 136 in receiver member 110. Additionally, in such embodiments, elongated member 104 can be received in upper portion 136b of channel 136 adjacent an upper surface 162 of crown member 160. In the illustrated embodiment, crown member 160 includes a spherical lower surface 164 corresponding to and configured to fittingly engage spherical upper surface 122 of head portion 118 of fixation element 112. Additionally, in certain embodiments, crown member 160 can define a hole 166 extending therethrough, corresponding to tool-receiving bore 124 disposed in head portion 118 of fixation element 112. In such embodiments, hole 166 can be generally aligned with tool-receiving bore 124 to allow for the passage of an instrument through hole 166 to reach tool-receiving bore 124.

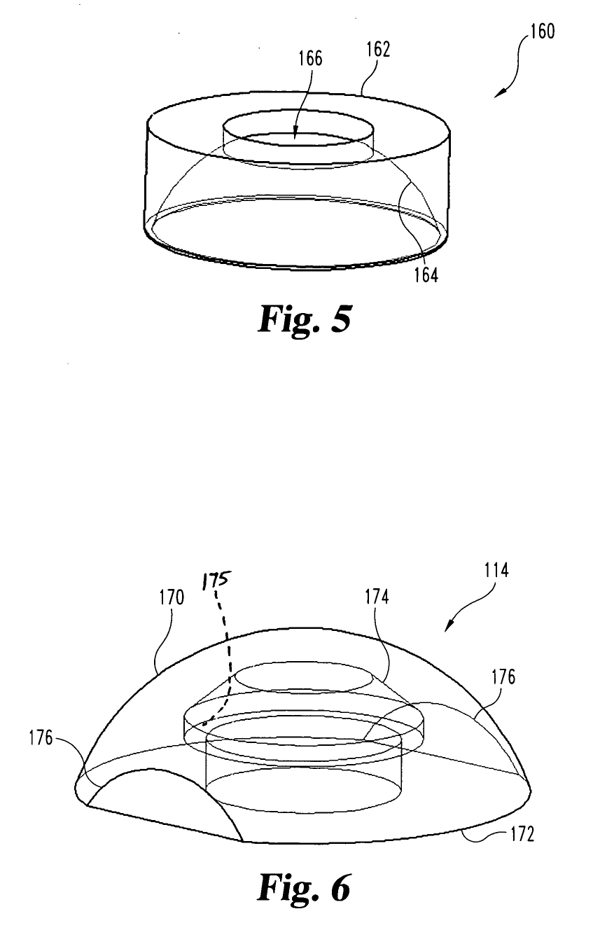

FIG. 6 illustrates an embodiment of snap-fit cap 114 according to the present disclosure. In the illustrated embodiment, cap 114 includes a spherical upper surface 170 and a lower surface 172. The smooth, spherical contour of surface 170 may help to reduce the likelihood of tissue irritation or damage caused by the implantation of system 100 in a patient. Additionally, cap 114 may define a bore or cavity 174 corresponding in configuration to the joined configuration of snap projections 142 and 144, e.g. including a groove or interior space 175. However, it should be appreciated that snap projections 142 and 144, and cavity 174, can be configured differently than as in the illustrated embodiment, such that cavity 174 generally defines the joined configuration of projections 142 and 144. Cap 114 is configured and operable to engage receiver member 110 via a snap-fit engagement so as to maintain receiver member 110 in a closed position (see FIG. 11), thereby capturing elongated member 104 in upper portion 136a of channel 136. In certain embodiments, cap 114 can further define diametrically opposing arcuate cut-out portions 176 configured to engage shoulders on an elongated member, as will be discussed in greater detail.

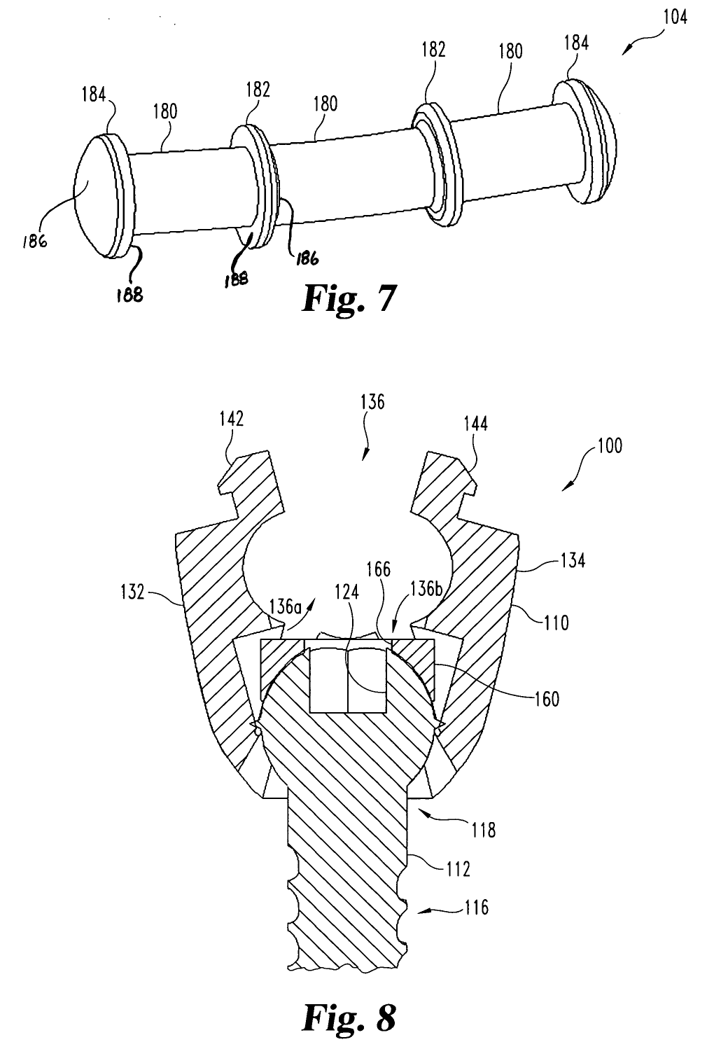

FIG. 7 illustrates an embodiment of an elongated member, such as spinal rod, which can be utilized with system 100. As illustrated, elongated member 104 can include a plurality of cylindrical segments 180 separated by middle shoulders 182 and terminating at end shoulders 184. Upon assembly of system 100, implants 102 can engage elongated member 104 at cylindrical segments 180. In the illustrated embodiment, shoulders 182 and 184 have a rounded side 186 and a substantially flat side 188, and an outer diameter larger than the diameter of adjacent segments 180, such that shoulders 182 and 184 substantially prevent translation of elongated member 104 relative to implants 102. Shoulders 182 and 184 may be unitarily formed with elongated member 104, or may be separately made and placed on elongated member 104. In the latter case, shoulders 182 and 184 may be of biocompatible metal and welded, glued, interference fitted, or otherwise joined to elongated member 104. They may also be made of flexible, elastic or compressible biocompatible materials (e.g. rubbers, plastics) and fitted or fixed onto elongated member 104 so that they are substantially or completely immobile with respect to elongated member 104.

In certain embodiments, part or all of system 100 is substantially radiolucent such that visibility of system 100 upon application of a radiation-based technique is substantially reduced. In certain embodiments, at least one of elongated member 104, receiver member 110, cap 114 and crown member 160 is composed of a biocompatible, non-metallic radiolucent material, and all such parts could be so made. It is also contemplated that elongated member 104, receiver member 110, cap 114 and/or crown member 160 can be composed of biocompatible materials such as polyetheretherketone (“PEEK”), carbon fiber or carbon fiber impregnated PEEK. Additionally, in certain embodiments, fixation element 112 can be composed of a metallic material, such as titanium or stainless steel, or carbon fiber impregnated PEEK. In certain other embodiments, the various components of system 100 may be composed of biocompatible ceramics or plastics, or other such appropriate materials.

Generally referring to FIGS. 1-12, the assembly, operation and use of system 100 will be described with reference to a surgical procedure involving a section of spine of a patient. It should be appreciated that other uses of system 100 described herein and other surgical procedures can be made.

In certain embodiments, partial assembly of system 100 occurs prior to implantation of system 100 into the patient. With receiver member 110 in an open position, fixation element 112 can be engaged therewith by inserting threaded bone-engaging portion 116 through hole 146 in receiver member 110, to a position where head portion 118 of fixation element 112 is received in lower portion 136b of channel 136 (see FIG. 8). Additionally, either before or after engagement of fixation element 112 with receiver member 110, crown member 160 can be positioned adjacent head portion 118 of fixation element 112, such that hole 166 may be substantially aligned with bore 124 in head portion 118. It is contemplated that in other embodiments, at least partial assembly of system 100 can occur during the surgical procedure and/or substantially simultaneously with implantation of part of system 100.

To treat the condition or injury of the patient, the surgeon obtains access to the surgical site in any appropriate manner, e.g. through incision and retraction of tissues. It is contemplated that system 100 discussed herein can be used in minimally-invasive surgical techniques where the spinal segment is accessed through a micro-incision, a sleeve, or one or more retractors that provide a protected passageway to the area. System 100 discussed herein also has application in open surgical techniques where skin and tissue are incised and retracted to expose the surgical site.

Once access to the surgical site has been obtained, e.g. via an opening such as a midline incision above the affected area, with tissue being resected, or by other surgical procedure, the surgeon may implant one or more implants 102 discussed herein adjacent vertebrae of a spinal segment that require compression, distraction and/or support in order to relieve or improve their condition. In certain embodiments, pilot holes in vertebrae may be made and threaded bone-engaging portions 116 of the fixation elements 112 may be inserted into or otherwise connected to a vertebral body. Bone engaging portions 116 can be threaded into the vertebrae to a desired depth and/or desired orientation relative to a longitudinal axis of the spinal segment. In certain embodiments, the surgeon or other medical professional can use a driving tool or other similar instrument by inserting the head of the instrument through hole 166 in crown member 160 (if present), through hole 146 of receiver member 110 and into engagement with tool-receiving bore 124 of fixation element 112 to drive threaded bone-engaging portion 116 into the vertebra.

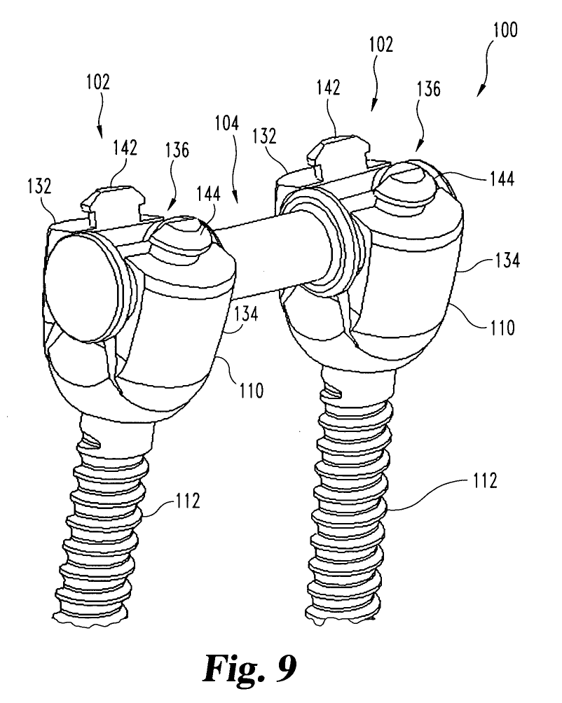

In an open position, receiver member 110 may be positioned in any of a number of angular positions relative to fixation element 112 and, in certain embodiments, may be rotated about head portion 118 of fixation element 112 to a desired position. In certain embodiments, crown member 160 can also be positioned in any of a number of positions relative to head portion 118 of fixation element 112, and can be generally aligned with receiver member 110. Once receiver member 110 is at the desired position relative to fixation element 112, elongated member 104 (e.g. a segment 180) can be placed in upper portion 136a of channel 136 adjacent upper surface 162 of crown member 160 (see FIG. 9). In the illustrated embodiment, channel 136 is open such that member 104 is loaded into channel 136 in a direction generally parallel to longitudinal axis L2 of hole 146. Further, member 104 can be received in another implant, such as a second implant 102, inserted into another vertebra to secure a section of vertebrae of a spinal segment. In many instances of spinal surgery, a surgeon will orient elongated members, such as member 104, so that the members are positioned substantially parallel to a portion of the spine. In certain embodiments, once any and all adjustments are made to reach the proper and desired positioning of the selected elongated member and orthopedic implant, the implant can be secured in the desired position.

Once receiver member 110 is at the desired position relative to fixation element 112 and elongated member 104 is positioned in channel 136 as desired, receiver member 110 can be locked at the selected position. In the illustrated embodiments, locking receiver member 110 includes transitioning receiver member 110 to a closed position by moving branches 132 and 134 towards each other to substantially surround a segment of elongated member 104 (see FIG. 10). In some embodiments, when receiver member 110 is in a closed position, barbs 120 disposed on head portion 118 of fixation element 112 will embed into or otherwise grip one or both of branches 132 and 134 of receiver member 110. The embedding of barbs 120 into receiver member 110 limits or prevents motion of receiver member 110 relative to fixation element 112 and secure receiver member 110 at the desired angular position (see FIG. 11).

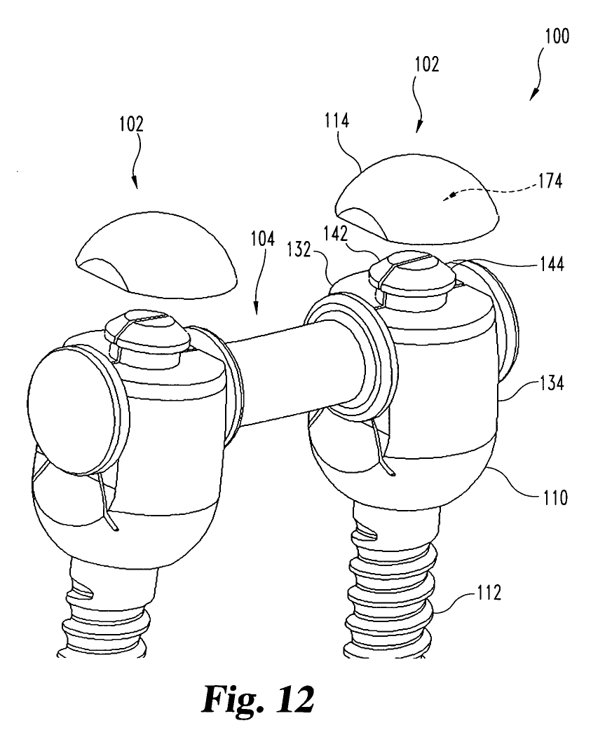

Cap 114 can be snapped onto projections 142 and 144 of receiver member 110, such that projections 142 and 144 are fittingly received in cavity 174 of cap 114 (see FIG. 12). The conical internal surfaces in the illustrated embodiment of cap 114 can exert pressure on the conical surfaces of projections 142 and 144 to help move branches 132 and 134 together. Accordingly, cap 114 may be configured to maintain receiver member 110 in a closed position, thereby retaining elongated member 104 in channel 136. In the illustrated embodiment, cap 114 exerts substantially no force on elongated member 104, such that no interference fit is created between elongated member 104 and receiver member 110, and accordingly at least slight movement of member 104 in channel 136 is allowed. Such dynamic connections can have substantial advantages in healing. In certain embodiments, at least slight rotation and other movement perpendicular to longitudinal axis L1 of channel 136 of elongated member 104 may be allowed to preserve motion of the spinal segment. Additionally, in certain embodiments, some or all translation of elongated member 104 along longitudinal axis L1 of channel 136 is substantially prevented by shoulders 182 and 184 on member 104.

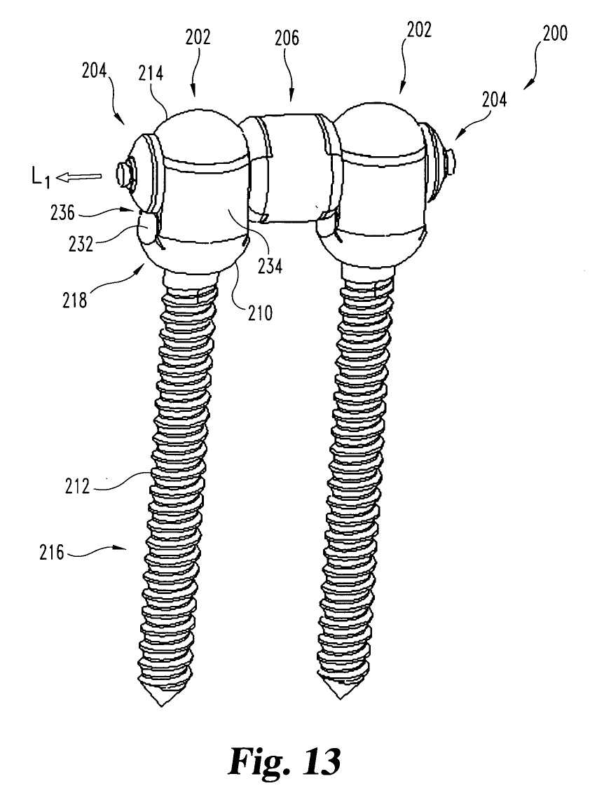

Referring generally to FIGS. 13-19, there is shown an embodiment of a spinal fixation system 200, in which implants 202, similar to implants 102, are connected with one or more elongated members 204, such as spinal rods, and further includes a resilient bumper 206 engaged with members 204 and positioned between implants 202. Each implant 202 can be inserted into a vertebra of a spinal segment of a patient and members 204 can be utilized to maintain one or more vertebrae of the spinal segment at a desired position. In certain embodiments, system 200 may include three or more implants 202 connecting at least one elongated member 204, with at least two resilient bumpers 206 positioned between the implants.

Bumper 206 can be utilized to maintain a separation distance between implants 202, prevent translation of members 204 with respect to each other (if more than one member 204 is present) or to implants 202, and/or provide resiliency during movement of system 200. The figures illustrate bumper 206 used in conjunction with implants 202 and elongated members 204 for illustration purposes only. It should be appreciated that bumper 206 can be used in conjunction with various other appropriate bone anchors or bone-engaging mechanisms, various other appropriate elongated members, and/or as part of various other spinal fixation systems. To that end, the elongated members utilized in conjunction with system 200 can be any appropriate elongated members such as bars, connectors, or other orthopedic constructs.

In certain embodiments, implant 202 can include a connector or receiver member 210 in engagement with a fixation element 212 and a snap-fit cap 214, similar or identical to those described above in connection with implant 102. As illustrated in FIG. 3, fixation element 212 can include a threaded bone-engaging portion 216 configured to engage bone and a head portion 218 configured to be received in receiver member 210. In certain embodiments, receiver member 210 includes branches 232 and 234 defining a U-shaped channel 236 with a longitudinal axis L1, with member 204 positioned in channel 236 and extending generally along longitudinal axis L1. Additionally, similar to cap 114, snap-fit cap 214 may be configured to snap-fit to receiver member 210 to maintain receiver member 210 in a closed position, capturing member 204 in channel 236.

In certain embodiments, members 204, receiver member 210 and cap 214 are smoothly-contoured to reduce the likelihood of tissue irritation and/or other tissue damage upon implantation of system 200 in a patient. The components of implant 202 are similar in structure and function to the components of implant 102, and therefore the details of implant 202 will not be included for the sake of brevity. It should be appreciated that system 200 can be anchored to vertebrae through different means, including through the use of hooks, clamps, bolts or other such appropriate fixation members for securing the system at proper and desired locations and orientations.

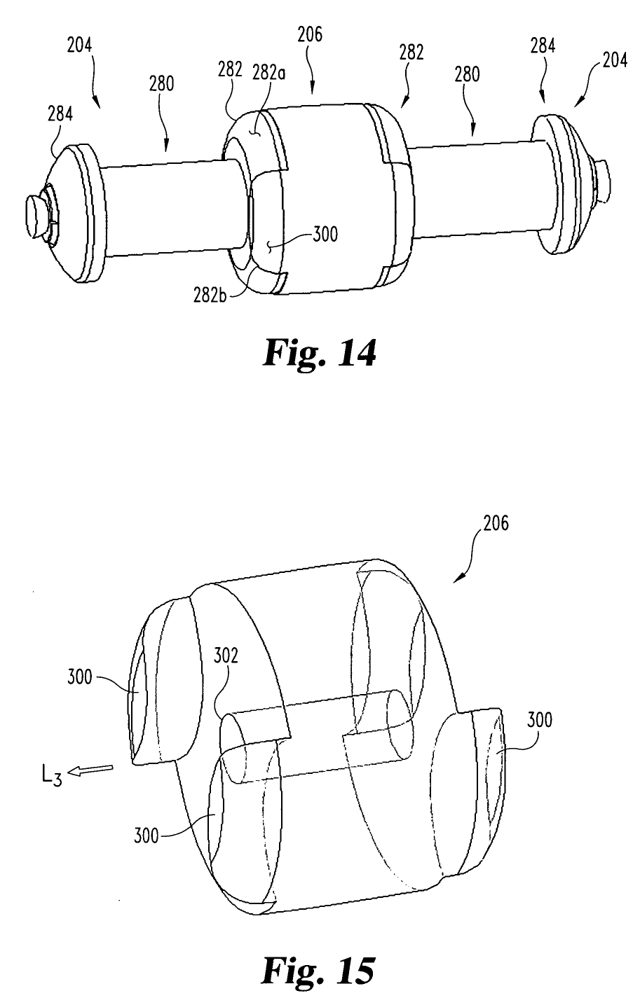

FIG. 14 illustrates bumper 206 in engagement with elongated members 204, such as spinal rods, with can be utilized with system 200. In the illustrated embodiment, two elongated members 204 are engaged with bumper 206. However, it should be appreciated that a single elongated member can be engaged with bumper 206 for use within system 200. In the illustrated embodiment, members 204 include a cylindrical segment 280 with an engagement shoulder 282 positioned at one end and an end shoulder 284. Shoulder 282, as illustrated, can be generally disk-like in shape with alternating extension portions 282a and cut-away portions 282b. In certain embodiments, cut-away portions 282b of shoulder 282 may receive tabs 300 of bumper 206 to engage member 204 with resilient bumper 206. However, it should be appreciated that the elongated members can be configured differently and can engage resilient bumper 206 in other appropriate manners. In the illustrated embodiment, end shoulder 284 is generally disk-like in shape, having a rounded surface with a decreasing diameter in a direction away from shoulder 282 and an opposite substantially flat surface. Shoulders 282 and 284 both include sections with a diameter larger than the diameter of adjacent segment 280, such that shoulders 282 and 284 may substantially limit or prevent translation of elongated member 204 relative to implants 202. In certain embodiments, the smooth, rounded surfaces of segments 280 and shoulders 282 and 284 may assist in reducing the likelihood of tissue irritation and damage upon implantation of system 200 in a patient.

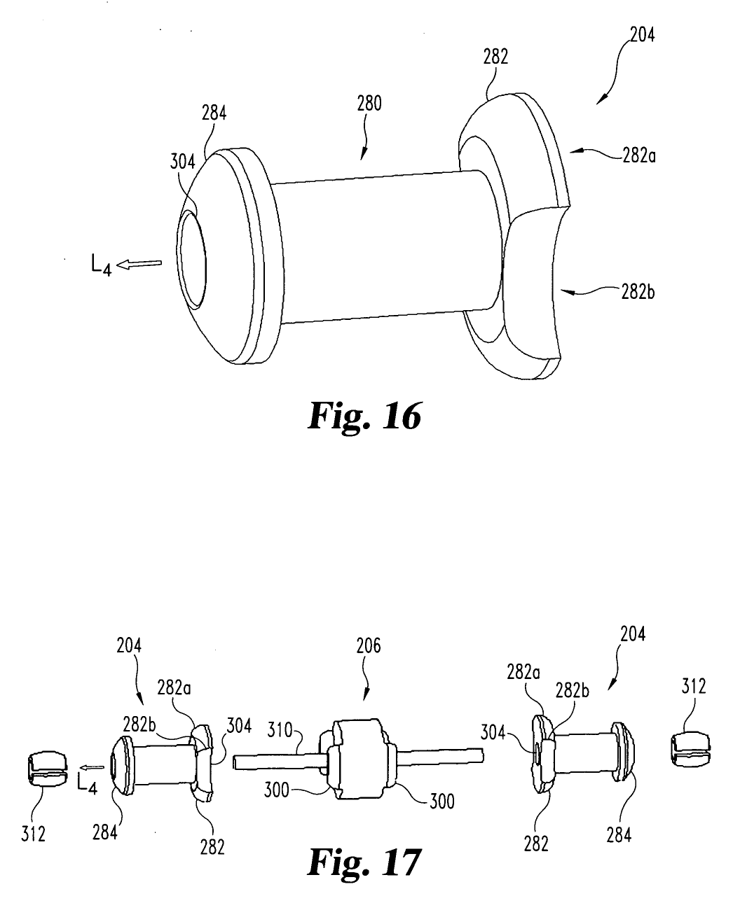

FIG. 15 illustrates resilient bumper 206, smoothly contoured to reduce the likelihood of tissue irritation or other tissue damage upon implantation of system 200 in a patient. In certain embodiments, bumper 206 can include tabs 300 configured to be received in cut-away portions 282b of shoulders 282 and mate with extension portions 282a to engage resilient bumper 206 with members 204. The illustrated embodiment includes two tabs 300 on each side of resilient bumper 206; however, it should be appreciated that bumper 206 can include more or fewer tabs as desired. Additionally, in certain embodiments it is contemplated that tabs 300 are absent and resilient bumper 206 engages members 204 in other appropriate manners. Additionally, as illustrated in FIG. 15, bumper 206 can define a hole 302 extending along a longitudinal axis L3 through resilient bumper 206, to assist in engaging members 204 with resilient bumper 206, as will be discussed in greater detail. Similarly, as illustrated in FIG. 16, member 204 can define a hole 304 extending along a longitudinal axis L4 from shoulder 282 to shoulder 284, to further assist in engaging members 204 with resilient bumper 206. In certain embodiments, longitudinal axes L3 and L4 may be aligned when system 200 is assembled.

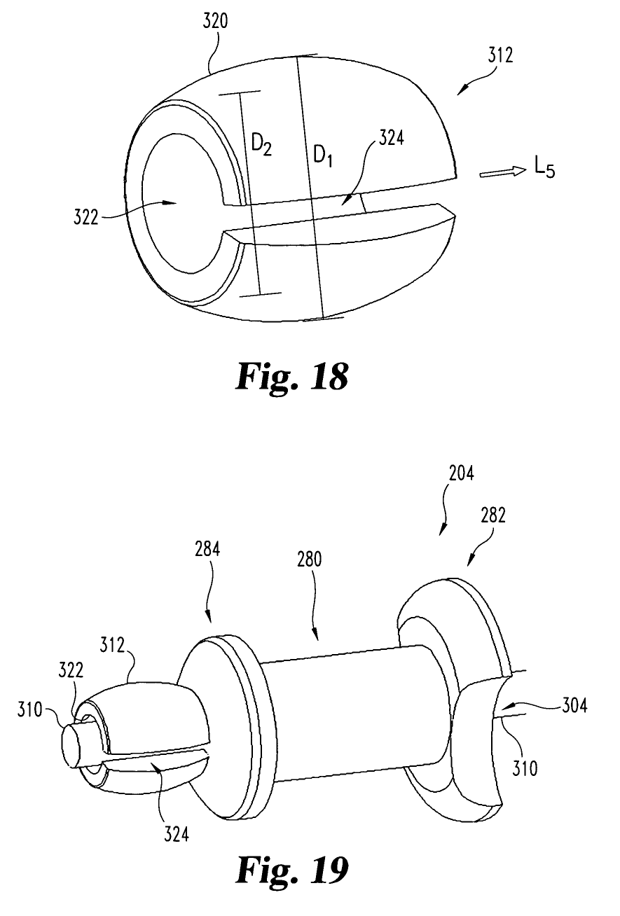

FIG. 17 is an exploded view of various components of system 200 to better illustrate some components of the system. As illustrated, in certain embodiments, system 200 can include a tether 310 configured for passage through hole 302 in bumper 206 and holes 304 in members 204. Additionally, system 200 can include at least two ferrules 312 configured for engagement with ends of tether 310 to secure members 204 in engagement with bumper 206. There is illustrated in FIG. 18 an embodiment of a ferrule 312. In the illustrated embodiment, ferrule 312 is generally cylindrical or olive-shaped, and defines a body 320 defining a central passageway 322 extending along a longitudinal axis L5 to receive ends of tether 310. Body 320 of ferrule 312 can also define a gap 324 to allow for at least slight compression of ferrule 312. Ferrule 312 can include an unstressed or natural outer diameter D1, i.e. a diameter measured when ferrule 312 is under no contractive (gap-closing) stress, and a compressed diameter D2, i.e. a diameter measured when ferrule 312 is experiencing contractive (gap-closing) stress. In certain embodiments, longitudinal axes L3, L4 and L5 may be aligned when system 200 is assembled.

In certain embodiments, part or all of system 200 is substantially radiolucent such that visibility of system 200 upon application of a radiation-based technique is substantially reduced. In certain embodiments, at least one of elongated member 204, receiver member 210, cap 214 and resilient bumper 206 is composed of a biocompatible, non-metallic radiolucent material. In some embodiments, resilient or flexible bumper 206 may be composed of polycarbonate urethane. Additionally, in some embodiments, tether 310 may be composed of polyethylene. Further, in some embodiments, ferrules 312 may be composed of non-metallic materials. It is contemplated that implant 202 and elongated member 204 can be composed of the materials described above in connection with implant 102 and member 104, respectively.

Generally referring to FIGS. 13-20, the assembly, operation and use of system 200 will be described with reference to a surgical procedure involving a section of spine of a patient. It should be appreciated that other uses of system 200 described herein and other surgical procedures can be made.

In certain embodiments, part of the assembly, operation and use of system 200 follows the assembly, operation and use of system 100. Therefore, for the sake of brevity, much of the discussion regarding implants 202 will not be included with reference to the discussion of system 200, as the implantation and locking of implants 202 can be performed in similar manners as described above in connection with the implantation and locking of implant 102. Similar to system 100, partial assembly of system 200 may occur prior to implantation of system 200 into the patient. In certain embodiments, fixation element 212 is engaged with receiver member 110 substantially as described above in connection with fixation element 112 and receiver member 110.

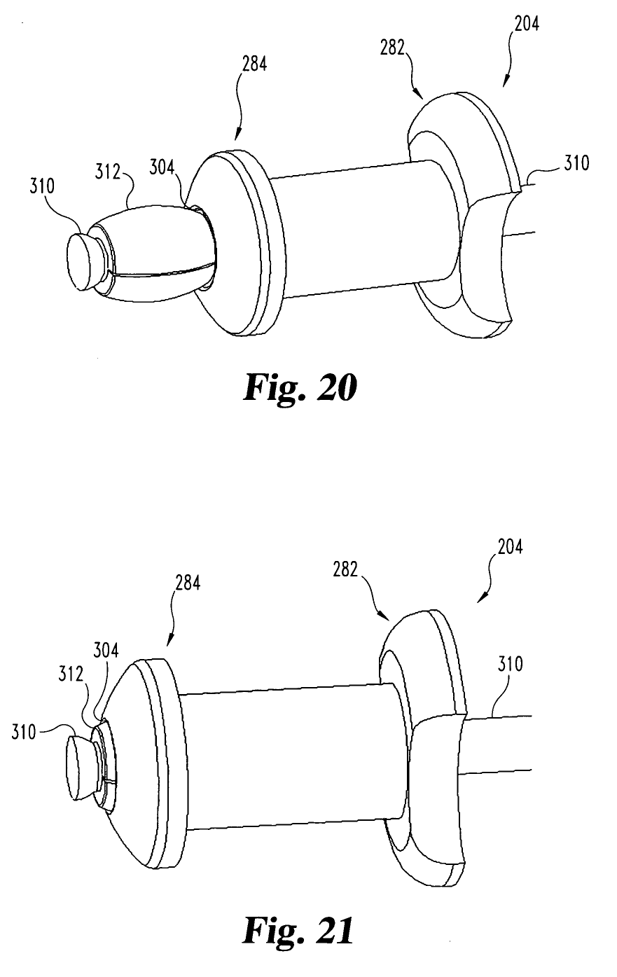

In certain embodiments, elongated members 204 may be engaged with resilient bumper 206 via tether 310 and ferrules 312. However, it should be appreciated that resilient bumper 206 can be engaged with one or more elongated members in various other manners. Initially, tether 310 may be inserted through hole 302 in bumper 206. Tether 310 can then be inserted through hole 304 in elongated member 204 and through passageway 322 of ferrule 312 (see FIG. 19). Ferrule 312 may be closed around tether 310 by contracting ferrule 312 and thereby reducing gap 324 created by body 320 of ferrule 312 (see FIG. 20). Ferrule 312, positioned about a portion of tether 310, may be inserted into hole 304 in elongated member 204 proximate shoulder 284. The insertion of ferrule 312 in hole 304 may further contract ferrule 312, lessening gap 324 and urging ferrule 312 toward compressed diameter D2. In such embodiments, contraction of ferrule 312 may cause ends of tether 310 to bulge outward, as illustrated in FIGS. 20 and 21. In other embodiments, end caps may be placed on the ends of tether 310 to further secure tether 310 and ferrule 312 in hole 304. In certain embodiments, body 320 of ferrule 312 may naturally or automatically urge ferrule 312 to expand toward natural diameter D1, creating an interference fit between ferrule 312 and member 204 and thereby limiting further movement of ferrule 312 in hole 304. In such embodiments, ferrule 312 resides in hole 304 (see FIG. 21) to engage elongated member 204 with bumper 206.

Either before or after engagement of ferrule 312 with tether 310, shoulder 282 of elongated member 204 may be engaged with bumper 206 via portions 282a and 282b of elongated member 204 and tabs 300 disposed on resilient bumper 206. In such embodiments, tabs 300 may be received in cut-away potions 282b to mate with extension portions 282a. In certain embodiments, engagement of two elongated members 204 with bumper 206 can occur substantially simultaneously. In certain embodiments, engagement of one elongated member 204 with bumper 206 may occur substantially prior to engagement of the other of elongated member 204 with bumper 206.

To treat the condition or injury of the patient, the surgeon obtains access to the surgical site in any appropriate manner, e.g. through incision and retraction of tissues. It is contemplated that system 200 discussed herein can be used in minimally-invasive surgical techniques where the spinal segment is accessed through a micro-incision, a sleeve, or one or more retractors that provide a protected passageway to the area. System 200 discussed herein also has application in open surgical techniques where skin and tissue are incised and retracted to expose the surgical site.

Once access to the surgical site has been obtained, e.g. via an opening such as a midline incision above the affected area, with tissue being resected, or by other surgical procedure, the surgeon may implant one or more implants 202 discussed herein adjacent vertebrae of a spinal segment that require compression or distraction in order to relieve or improve their condition. The implants 202 may be implanted substantially in the same manner as described in connection with the implantation of implants 102. Thereafter, elongated members 204 in engagement with bumper 206 may be engaged with receiver member 210 in a substantially similar manner as described above in connection with the engagement of elongated member 104 with receiver member 110, to complete the implantation of system 200 in a patient. However, it should be appreciated that resilient bumper 206 can be engaged with other elongated members and/or orthopedic implants as desired by a user of the system.

While the disclosure has been illustrated and described in detail in the drawings and foregoing description, the same is to be considered as illustrative and not restrictive in character. It should be understood that only certain embodiments have been shown and described and that all changes and modifications that come within the spirit of the disclosure are desired to be protected.

What is claimed is:

1. An orthopedic apparatus, comprising:
an orthopedic implant device having a receiver member and a fixation element member;
wherein said receiver member includes first and second branches defining a channel therebetween, each of said branches having a top surface, wherein said channel extends along a longitudinal axis and is configured to receive an elongated member, wherein said fixation element member includes a threaded portion configured to engage bone; and
a cap configured to snap fit to said receiver member proximal said top surfaces to substantially close at least part of said channel and secure the elongated member in said channel.

2. The apparatus of claim 1, wherein said fixation element member includes a head portion and said receiver member is configured to engage said head portion.

3. The apparatus of claim 2, wherein said receiver member is positionable in a first open configuration and a second closed configuration, said head portion being moveably connected with said receiver member when said receiver member is in said first open configuration, said head portion being fixedly engaged to said receiver member when said receiver member is in said second closed configuration, thereby locking said receiver member at a desired angular position relative to said fixation element member.

4. The apparatus of claim 3, wherein said head portion includes barbs configured to embed into said receiver member when said receiver member is positioned in said second closed configuration.

5. The apparatus of claim 2, comprising a crown member having an upper surface and a lower surface, wherein said lower surface of said crown member is configured to engage said head portion, and wherein said receiver member is configured to engage said crown member such that the elongated member is positionable adjacent said upper surface crown member.

6. The apparatus of claim 5, wherein said head portion is at least partially spherical and said lower surface of said crown member is at least partially spherical corresponding to said head portion.

7. The apparatus of claim 1, wherein said receiver member includes a projection extending from each of said top surfaces and said cap defines a bore configured to receive said projections, wherein said cap is configured to snap onto said receiver member, with said projections snapping into said bore, to secure the elongated member in said channel.

8. The apparatus of claim 1, wherein said orthopedic implant device is a bone screw.

9. The apparatus of claim 8, wherein said bone screw is multi-axial and said receiver member is positionable in any one of a plurality of angular positions relative to said fixation element member.

10. The apparatus of claim 1, wherein the apparatus is radiolucent.

11. The apparatus of claim 1, wherein said receiver member and said cap are each composed of a non-metallic material.

12. The apparatus of claim 1, wherein said receiver member is positionable in a first open position to receive the elongated member and a second closed position to substantially capture the elongated member in said channel.

13. An orthopedic system, comprising:
at least two bone screws, wherein each bone screw includes a threaded bone-engaging portion;
at least two corresponding receiver members, wherein each receiver member includes two branches defining a channel configured to receive an elongated member, each branch including a top surface;
at least one elongated member configured to be received in said channels;
at least two snap-fit caps, wherein each cap is configured to snap fit to said corresponding receiver member proximal said top surfaces to substantially close at least part of said channel and capture said elongated member in said channel.

14. The system of claim 13, comprising at least two crown members, wherein said crown members are configured to engage said bone screws, with said crown members received in said corresponding receiver members.

15. The system of claim 13, wherein said elongated member includes at least one shoulder to prevent translation of said elongated member relative to said receiver members.

16. The system of claim 13, comprising a bumper positionable between said receiver members, wherein said bumper is configured to engage with said elongated member.

17. The system of claim 13, wherein said receiver members and said snap-fit caps are radiolucent.

18. An orthopedic implant, comprising:
a fixation element including a threaded portion configured to engage bone and a head portion;
a receiver member engageable with said head portion of said fixation element, said receiver member including first and second branches defining a channel therebetween, wherein said channel extends along a longitudinal axis and is configured to receive an elongated member; and
a locking mechanism configured to substantially capture the elongated member in said channel;
wherein said receiver member is positionable in a first open position to receive an elongated member, the first open position including a first gap between said branches, and a second closed position to substantially enclose an elongated member in said channel, said second closed position including a second gap between said branches, wherein the first gap is larger than the second gap.

19. The implant of claim 18, wherein said locking mechanism includes a snap-fit cap configured to snap to said receiver member when said receiver member is in said second closed position.

20. The implant of claim 18, wherein the implant is radiolucent.

21. The implant of claim 18, wherein said head portion is moveably engaged with respect to said receiver member when said receiver member is in said first open position, and wherein said head portion is fixedly engaged to said receiver member when said receiver member is in said second closed position, thereby locking said receiver member at a desired angular position relative to said fixation element.

22. The implant of claim 21, wherein said head portion includes barbs configured to embed into said receiver member when said receiver member is positioned in said second closed position.

23. A system, comprising:
at least two orthopedic implant devices each having a receiver member and a fixation element member, wherein each of said receiver members is configured to receive a portion of an elongated member, wherein each of said fixation element members includes a threaded portion configured to engage bone;
at least one elongated member configured to be received in said receiver members; and
a resilient bumper positionable between said receiver members, wherein said bumper is configured to engage said at least one elongated member.

24. The system of claim 23, wherein said at least one elongated member includes two elongated members, each including a first end configured to engage said bumper.

25. The system of claim 24, comprising a tether extending through said bumper and said elongated members, wherein said bumper and said elongated members each define a hole configured for passage of said tether.

26. The system of claim 25, comprising at least two ferrules, with one of said ferrules positionable in said hole in one of said elongated members and another of said ferrules positionable in said hole in the other of said elongated members, wherein each of said elongated members includes a second end opposite said first end, and wherein each of said ferrules includes an olive-shaped configuration with a gap to allow for compression of said ferrule around said tether and insertion of said ferrule in said corresponding hole at said corresponding second end to maintain engagement of said elongated members and said bumper.

27. The system of claim 23, wherein said bumper is composed of polycarbonate urethane.

28. A method, comprising:
providing a fixation element in engagement with a receiver member, wherein said receiver member includes first and second branches defining a channel configured to receive an elongated member, wherein said fixation element includes a threaded bone-engaging portion;
engaging said threaded bone-engaging portion of said fixation element to a vertebra;
placing an elongated member in said channel;
providing a snap-fit cap configured to engage said receiver member via a snap-fit engagement; and
snapping said cap to said receiver member to capture the elongated member in said channel.

29. The method of claim 28, comprising pivoting said receiver member to a desired angular position relative to said fixation element.

30. The method of claim 29, comprising closing said branches of said receiver member to lock said receiver member at said desired angular position relative to said fixation element, wherein said closing occurs prior to said snapping.

31. The method of claim 30, wherein said fixation element includes at least one projection configured to embed into said receiver member upon said closing of said branches to lock said receiver member at said desired angular position relative to said fixation element.

32. The method of claim 28, comprising positioning a crown member on said fixation element, wherein said receiver member engages said crown member and the elongated member is positioned adjacent said crown member.

33. The method of claim 28, wherein said branches each include a top surface and a projection extending from said top surface, wherein said cap defines a bore corresponding to the collective configuration of said projections such that said cap is configured to snap onto said receiver member.
